SEQUENCE LISTING (6 sequences)

SEQ ID NO: 1 - DNA, 1119 bp, Trichosporonoides megachiliensis
  CDS (1)..(993)
atgtcttacaaacagtacatccccctgaacgacggtaacaaaatccctgcccttggatttggtacttggcaagctgaacctggtcaagtgggtgcaagtgtcaagaacgctgtcaaggctgggtaccgtcatttggatttggccaaagtgtaccaaaaccaatcggaaattggagtagcacttcaggaactgtttgatcaaggtattgttaaacgggaagatttgtttattacgtccaaagtatggaataaccgtcatgctcctgaacatgttgagcctgcattggacgaaacattgaaagaacttggattgtcctacttggatttgtacttgattcattggcccgttgcgttcaagtttactacgcctcaagaactattccctactgagccggataacaaggaattggccgcgattgatgattcaatcaagttggtagacacttggaaggcagttgtagcactcaaaaaaacgggtaagaccaaatccgttggtgtgtcgaacttcactacggatttggtagacttggttgaaaaagcgtcgggggaacgaccggcggtcaatcagattgaagcacacccattgttacaacaggatgaattggttgctcatcacaagagtaaaaacattgtgattactgcgtacagtcctttgggaaacaatgtgagtgggaaaccacctctgactcaaaaccctgggattgaagcaactgcgaaacggttaaatcatactcctgctgcggtcttgcttgcatgggggattcaacgtggatacagtgtattggtcaagagtgttacaccttctcgaattgagagcaattatgatcagattaccctttctcctgaagaattccagaaggttacggatttgatcaaggaatatggcgaaagtcgcaacaatattccgttgaattataaaccttcatggcccatcagtgtgtttggtacatcggatgaagctaaggctactcataagattaacaccaacctttgagttcagtttgggaactatttaaagctgcttgctggtcacattattgtcagtacctaccatgaagaattcaatattattttacattgtcaaccattacatggatccaaaaaaaaaaaaaaaaaaaaa

SEQ ID NO: 2 - protein, 330 aa, Trichosporonoides megachiliensis
MSYKQYIPLNDGNKIPALGFGTWQAEPGQVGASVKNAVKAGYRHLDLAKVYQNQSEIGVALQELFDQGIVKREDLFITSKVWNNRHAPEHVEPALDETLKELGLSYLDLYLIHWPVAFKFTTPQELFPTEPDNKELAAIDDSIKLVDTWKAVVALKKTGKTKSVGVSNFTTDLVDLVEKASGERPAVNQIEAHPLLQQDELVAHHKSKNIVITAYSPLGNNVSGKPPLTQNPGIEATAKRLNHTPAAVLLAWGIQRGYSVLVKSVTPSRIESNYDQITLSPEEFQKVTDLIKEYGESRNNIPLNYKPSWPISVFGTSDEAKATHKINTNL

SEQ ID NO: 3 - DNA, 20 bp, Artificial Sequence
  synthetic DNA
cargarctnttygaycaygg

SEQ ID NO: 4 - DNA, 20 bp, Artificial Sequence
  synthetic DNA
tgngcytcnatytgrttnac

SEQ ID NO: 5 - protein, 7 aa, Trichosporonoides megachiliensis
QELFDQG

SEQ ID NO: 6 - protein, 7 aa, Trichosporonoides megachiliensis
VNQIEAH

(19) United States
(12) Patent Application Publication
(10) Pub. No.: US 2002/0127677 A1
(43) Pub. Date: Sep. 12, 2002

(54) Erythrose reductase, its cDNA and cell which the cDNA express

(75) Inventors: Tetuya Ookura, Tsukuba-shi (JP); Takafumi Kasumi, Ushiku-shi (JP); Eiji Asaba, Omiya-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Director of National Food Research Institute, Ministry of Agriculture, Forestry and Fisheries, Tsukuba-shi (JP)

(21) Appl. No.: 09/800,487

(22) Filed: Mar. 8, 2001

(30) Foreign Application Priority Data
Jan. 9, 2001 (JP) 2001-001294

(51) Int. Cl. C12P 7/18; C12N 9/02; C07H 21/04; C12P 21/02; C12N 5/06

(52) U.S. Cl. 435/189; 435/69.1; 435/325; 435/320.1; 536/23.2

(57) ABSTRACT

A protein shown in (A) or (B) below:

(A) a protein having an amino acid sequence of SEQ. ID No. 1 in the Sequence Listing;

(B) a protein having an amino acid sequence of SEQ. ID No. 1 in the Sequence Listing, wherein the amino acid sequence includes substitution, deletion, insertion, addition or inversion of one or several amino acids and wherein the protein has an erythrose reductase activity; a DNA encoding the protein; a cell to which a DNA has been transferred in a manner such that the DNA is capable of expressing an erythrose reductase type III the DNA encodes; and a method for producing erythritol, comprising acting the erythrose reductase type III or a cell to which erythrose reductase type III has been transferred in a manner capable of expressing the erythrose reductase type III on D-erythrose and harvesting the produced erythritol.

### FIELD OF THE INVENTION

The present invention relates to a novel protein having an erythrose reductase activity, to a complementary DNA encoding the protein, to a method for producing a protein possessing an erythrose reductase activity, and to a method for producing erythritol.

The erythrose reductase is an enzyme that reduces erythrose with NADPH for producing erythritol and NADP+. The enzyme includes three kinds of isozymes, i.e., type I, type II and type III classified after differences in mobility on Native-PAGE and isoelectric focusing electrophoresis.

### BACKGROUND OF THE INVENTION

Erythritol is a high quality and very low-calorie sweetness. Erythritol is also noncariotic so that it is widely used as a sweetener for food and beverage.

Several microorganisims such as Trichosporonoides and Moniliella are widely used for industrial production of erythritol from glucose. Microorganisms such as those belonging to the genera Trichosporonoides, Moniliella, etc., are caused to act on a substrate such as glucose (cf. Japanese Patent Publication No. Hei 6-30591, Japanese Patent Publication No. Hei 6-30592, Japanese Patent Publication No. Hei 4-11189, Japanese Patent Publication No. Hei 4-635, Japanese Patent Kokai No. Hei 10-96, and Japanese Patent Kokai No. Hei 9-154589).

It has been reported that erythrose reductase type I,II and III are involved in producing eythritol at Trichosporonoides megachiliensis Strain SN-G42 (FERM BP-1430) (K. Tokuoka, et al., J. Gen. Appl. Microbiol., 38, 145-155 (1992)).

The metabolic pathway from glucose to erythritol in Trichosporonoides megachiliensis Strain SN-G42 is illustrated in FIG.1.

As illustrated in FIG. 1, glucose enters the Pentose Phosphate Shunt to produce erythrose-4-phosphate (Erythrose-4-P) after this sugar phosphate is metabolited to glucose-6-phosphate (Glc-6-P) or glyceraldehyde-3-phosphate by glycolysis.

The erythrose-4-phosphate is dephosphorylated to produce D-erythrose, D-erythrose, which gets reduced by NADPH to produce meso-eythritol.

Of such a series of reactions, the erythrose reductase type III catalyses the latter reductive reaction (i.e., the reaction in which erythrose gets reduced by NADPH to form meso-eythritol).

The reports on erythrose reductases only described their enzymological properties. The genetical analysis of these enzymes remains to be elucidated.

An object of the present invention is to provide an efficient method for the production of erythritol.

Another object of the present invention is to clarify the primary structures of enzyme having an erythrose reductase activity and to characterize a complementary DNA encoding the protein in order to establish an erythritol producing microorganism and to provide a method for utilizing them.

If it is successful in obtaining the DNA that encodes the protein having an erythrose reductase activity, a large amount of proteins can be produced by expressing the DNA in a cell such as Esherichia coli, yeast cell, etc., or the like means. This invention not only leads to mass production of erythritol but also is applied to development of mutant enzymes having higher erythritol productivity, cloning of DNA encoding related enzymes and the like by using genetic engineering techniques.

### SUMMARY OF THE INVENTION

The present inventors have made extensive research with view to achieving the above-described object and as a result, they have found a base sequence of DNA encoding a protein having an erythrose reductase activity. Said protein is produced by a microorganism belonging to the genus Trichosporonoides.

That is, the present inventors have first harvested an enzyme from the microorganism and purified it, partially decoding the amino acid sequence of a protein having an erythrose reductase activity produced by the microorganism belonging to the genus Trichosporonoides by peptide mapping, and preparing a probe based thereon.

By performing Northern hybridization of erythritol producing microorganism using this probe, the time when erythrose reductase type III highly expressed was identified. A cDNA library was prepared from mRNA at the time when expression level is highest.

Then, the cDNA library was screened with the above-described probe, the cDNA was converted from phage to plasmid and then the base sequence of DNA of a protein having an erythrose reductase activity was decoded.

Further, the cDNA of the protein having an erythrose reductase activity was expressed as a histidine Tag-fused protein.

The activity, substrate specificity, and the like of the thus-obtained recombinant protein were examined and as a result, it was confirmed that the recombinant protein had a substrate specificity similar to that of natural type erythrose reductase and also had an enzymatic activity of producing a sugar alcohol.

The present invention has been completed based on these findings.

That is, in a first embodiment, the present invention provides a protein shown in (A) or (B) below:

(A) a protein having an amino acid sequence of SEQ. ID No. 1 in the Sequence Listing;

(B) a protein having an amino acid sequence of SEQ. ID No. 1 in the Sequence Listing, wherein the amino acid sequence includes substitution, deletion, insertion, addition or inversion of one or several amino acids and wherein the protein has an erythrose reductase activity.

In a second embodiment, the present invention provides a DNA encoding a protein shown in (A) or (B) below:

(A) a protein having an amino acid sequence of SEQ. ID No. 1 in the Sequence Listing;

(B) a protein having an amino acid sequence of SEQ. ID No. 1 in the Sequence Listing, wherein the amino acid sequence includes substitution, deletion, insertion, addition or inversion of one or several amino acids and wherein the protein has an erythrose reductase activity.

In a third embodiment, the present invention provides the DNA as described in the above second embodiment, wherein the DNA comprises one shown in (a) or (b) below:

(a) a DNA containing a base sequence comprising at least nucleotides Nos. 1 to 398 out of the nucleotide sequence described in SEQ. ID No. 1 in the Sequence Listing.

(b) a DNA hybridizing with a base sequence comprising at least nucleotides Nos. 1 to 398 out of the nucleotide sequence described in SEQ. ID No.1 in the Sequence Listing or a probe prepared therefrom under a stringent condition and encoding a protein having an erythrose reductase activity.

In a fourth embodiment, the present invention provides the DNA as described in the above third embodiment, wherein the stringent condition is a condition under which washing is performed at a salt concentration corresponding to 2×SSC and 0.1% SDS at 60° C.

In a fifth embodiment, the present invention provides the DNA as described in the above second embodiment, wherein the DNA comprises a DNA shown in (c) or (d) below:

(c) a DNA containing a base sequence comprising at least nucleotides Nos. 408 to 1119 out of the nucleotide sequence described in SEQ. ID No. 1 in the Sequence Listing.

(d) a DNA hybridizing with a base sequence comprising at least nucleotides Nos. 408 to 1119 out of the nucleotide sequence described in SEQ. ID No.1 in the Sequence Listing under a stringent condition and encoding a protein having an erythrose reductase activity.

In a sixth embodiment, the present invention provides the DNA as described in the above fifth embodiment, wherein the stringent condition is a condition under which washing is performed at a salt concentration corresponding to 2×SSC and 0.1% SDS at 60° C.

In a seventh embodiment, the present invention provides a cell to which a DNA as described in any one of the above second to sixth embodiments has been introduced in a manner such that the DNA is capable of expressing an erythrose reductase type III.

In an eighth embodiment, the present invention provides a method for producing erythrose reductase type III, comprising the steps of culturing the cell as described in the above seventh embodiment in a medium to produce and accumulate erythrose reductase type III in a culture liquid and harvesting the erythrose reductase type III from the culture liquid.

In a ninth embodiment, the present invention provides a method for producing erythritol, comprising the steps of acting a protein having an erythrose reductase activity as described in the above first embodiment on D-erythrose and harvesting a produced erythritol.

In a tenth embodiment, the present invention provides a method for producing erythritol, comprising the steps of acting the cell as described in the above seventh embodiment on D-erythrose and harvesting a produced erythritol.

### BRIEF DESCRIPTION OF THE DRAWINGS

FIG. 1 is a schematic diagram illustrating pathway of erythritol biosynthesis from glucose in erythritol producing microorganism;

FIG. 2 shows the results of Northern hybridization, wherein Lane 1 shows the result on the product of 24 hours culture, Lane 2 shows the result on the product of 48 hours culture, Lane 3 shows the result on the product of 72 hours culture, and Lane 4 shows the result on the product of 96 hours culture;

FIG. 3 is a restriction enzyme map of cDNA encoding an erythrose reductase type III protein, wherein EcoR I, Ban I, and BamH I represent restriction enzymes, and the numeral in the brackets indicates the number of bases counted from the 5′ terminal; and

FIG. 4 is a graph showing the enzyme activity of recombinant erythrose reductase type III, wherein ♦, ●, and ▴ stand for specific activities (units/mg) at 37° C., 25° C., and20° C., respectively and ⋄, ◯, and Δ stand for total activities (unit) at 37° C., 25° C., and 20° C., respectively.

### DETAILED DESCRIPTION OF THE INVENTION

Hereinafter, the present invention will be described in detail.

The protein having an erythrose reductase activity of the present invention exists in yeast belonging to the genus Trichosporonoides which is an erythritol producing microorganism. Trichosporonoides megachiliensis is a typical yeast.

In the present invention, the DNA encoding a protein having an erythrose reductase activity can be prepared, for example, by purifying a protein having an enzyme activity obtained from an erythritol producing microorganism, partially decoding an amino acid sequence of the protein, preparing a probe from the partially decoded amino acid sequence based on the cDNA of erythrose reductase type III of the above-described microorganism, and then screening the cDNA library of the above-described microorganism using the probe, as established by the present inventors.

The present inventors first obtained erythrose reductase type III from an erythritol producing microorganism by a conventional process in order to prepare a probe for screening, purified the enzyme and then decoded the amino acid sequence of the protein.

To obtain erythrose reductase type III from the Trichosporonoides megachiliensis Strain SN-G42, the procedures described in H. Ishizuka, et al., Biosci. Biotech. Biochem., 56(6), 941-945, 1992 may be employed.

There can be used not only this procedure but also usual procedure for obtaining and purifying protein with a combination of centrifugal separation, dialysis, various kinds of chromatographies, etc.

The partial amino acid sequence from the purified erythrose reductase type III can be obtained by peptide mapping by a conventional method after digestion.

The amino acid sequence can be determined by Edman degradation and use of an automatic amino acid sequencer makes the determination more convenient.

Next, the present inventors carried out PCR reaction using a primer designed from the partially decoded amino acid sequence and the cDNA from erythritol producing microorganism as a template to prepare a probe.

The design of a primer based on the partially decoded amino acid sequence can be performed by a conventional method.

For example, referring to the amino acid sequences of the aldo-keto reductase families, parts of the partially decoded amino acid (cf. SEQ. ID Nos. 4 and 5 in the Sequence Listing) can be selected and sense primers and anti-sense primers (cf. SEQ. ID Nos. 2 and 3 in the Sequence Listing) can be designed from the respective sequences.

The cDNA of erythritol producing microorganism can be obtained by extracting RNA from the culture liquid of erythritol producing microorganism, preparing mRNA therefrom and carrying out reverse transcription reaction using the mRNA as a template.

For the extraction of RNA, it is convenient to use of TRIZOL (produced by Gibco BRL). Also, mRNA can be prepared conveniently by using DYNABEADS mRNA Purification Kit (produced by DYNAL).

Using the thus-obtained single strand cDNA as a template and the sense primer (cf. SEQ. ID No. 2 in the Sequence Listing) and antisense primer (cf. SEQ. ID No. 3 in the Sequence Listing) designed in advance, a probe was amplified by PCR reaction. In this manner, the present inventors succeeded in obtaining a cDNA fragment having a length of 398 bp as a PCR product.

The PCR product was ligated into a plasmid vector for transformation into Esherichia coli cells. Plasmids from transformant were analyzed by DNA sequencing using Dye Terminator and ABI 310A DNA sequencer (Perkin Elmer) for examining partial amino acid sequence of erythrose reductase type III.

As a result, it was confirmed that PCR product encodes a part of the erythrose reductase type III. The PCR product was used as a probe for the plaque hybridization as described below.

The fragment corresponds to the 184th to 582nd bases from the N-terminal of the base sequence described in SEQ. ID No. 1 of the Sequence Listing.

Subsequently, upon preparing the cDNA library of the erythritol producing microorganism, the present inventors studied in advance the time at which erythrose reductase type III mRNA highly expressed.

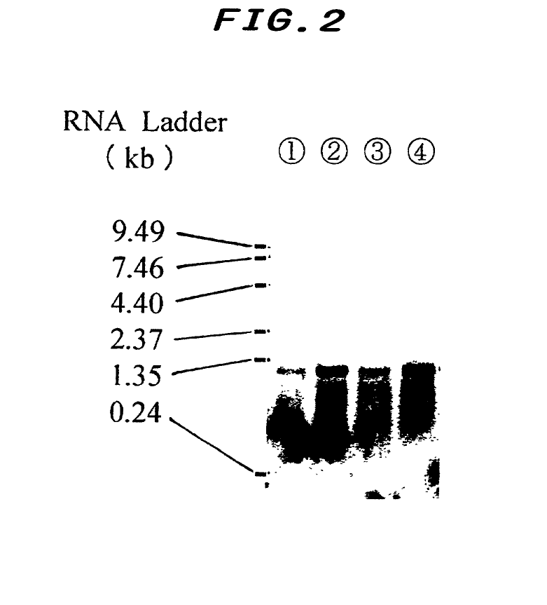

The method for examining the amount of expression of erythrose reductase type III includes Northern hybridization and the like methods. For example, total RNA was extracted from Trichosporonoides megachiliensis Strain SN-G42 cultivated for a varied time and Northern hybridization was carried out using the previously designed probe. As a result, it revealed that 48 hours cultivation product (Lane 3 in FIG. 2) showed the highest mRNA expression level for erythrose reductase type III.

Accordingly, the present inventors prepared a cDNA library of erythritol producing microorganism at the time when erythrose reductase type III mRNA highest expressed.

A cDNA library may be prepared by extracting RNA from a culture of erythritol producing microorganism, purifying mRNA, synthesizing double strand cDNA complementary thereto, and incorporating this into a phage vector with an adapter.

For the extraction of RNA, it-is convenient to use TRIZOL (produced by Gibco BRL). Also, mRNA can be prepared conveniently by using DYNABEADS mRNA Purification Kit (produced by DYNAL).

The cDNA library can be prepared by adopting Okayama-Burg method, Gubler-Hoffman method or the like. However, for the convenience's sake, the latter method is preferred.

In practice, it is preferable and convenient to use the method of preparing a library using ZAP Express cDNA Synthesis Kit (produced by STPATAGENE) according to the manufacture instruction.

ZAP Express Vector used in the kit of the invention is a linear phage DNA. However, this can be taken out by in vivo excision as a circular plasmid (phagemid) containing kanamycin-resistant gene.

The present inventors performed screening of the above-described cDNA library for DNA encoding erythrose reductase type III using the previously obtained probe.

The plaque hybridization was done using a probe labeled with digoxigenin.

The present inventors analyzed the sequence of DNA sequence after converting it to plasmid from phage positive to the probe as a result of screening.

For example, first, several plaques that were positive to the probe in screening were isolated and phage was amplified. Then, a phagemid portion containing an insert was excised out in vivo from the phage DNA. This was converted into the form of plasmid in order to make the handling easy and transfected to Escherichia coli to amplify the plasmid. Thereafter, DNA sequencing was performed on this plasmid by dideoxy method with ABI 310A DNA sequencer.

The present inventors have found a base sequence of a total length of 1119 bp shown in SEQ. ID No. 1 in the Sequence Listing by the DNA sequencing.

The amino acid sequence decoded from this DNA sequence is also shown in SEQ. ID No. 1 in the Sequence Listing. The protein having the amino acid sequence is the erythrose reductase type III protein.

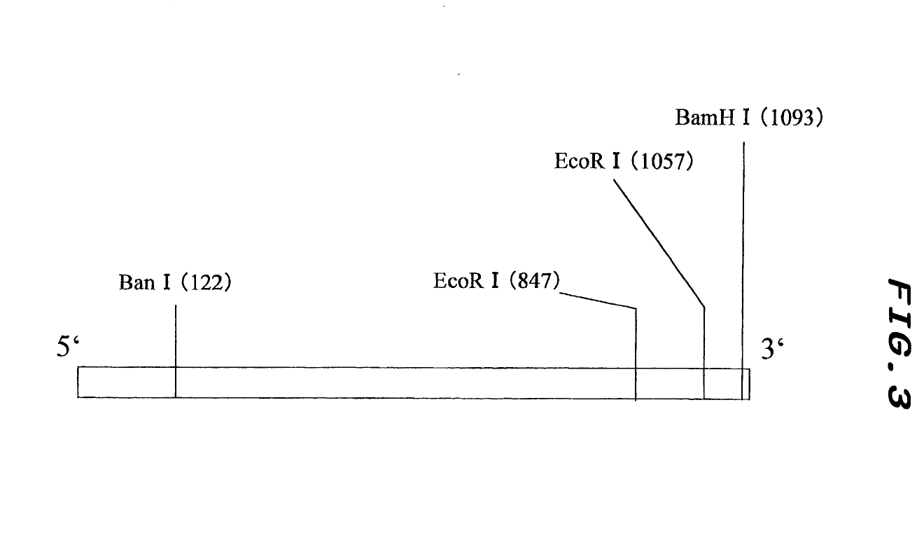

FIG. 3 shows a restriction enzyme map of the base sequence of DNA encoding erythrose reductase type III protein. As can be seen from FIG. 3, the base sequence has a Ban I cleavage site at the 122nd base from the 5′-terminal, EcoR I cleavage sites at 847th and 1057th bases from the 5′-terminal, and a BamH I cleavage site at the 1093rd base.

The amino acid sequence of the erythrose reductase type III protein (cf. SEQ. ID No. 1 of the Sequence Listing) is a novel amino acid having low homology with the previously clarified amino acid sequences of human aldose reductase and of yeast (Saccharomyces cerevisiae) gcy protein.

From this, it revealed that the erythrose reductase type III protein of the present invention had a novel amino acid sequence.

The protein having an erythrose reductase activity according to the present invention may comprise an amino acid sequence containing one or more substitution, deletion, insertion, addition or inversion at one or more sites with respect to the amino acid sequence of SEQ. ID No. 1 in the Sequence Listing, if erythrose reductase activity exists.

The protein that comprises the amino acid sequence containing one or more substitution, deletion, insertion, addition or inversion at one or more sites with respect to the amino acid sequence of SEQ. ID No. 1 in the Sequence Listing as such can be obtained, for example, by site specific mutation method (Methods in Enzymology, 100, pp. 448 (1983)), mutation treatment and in addition natural occurring mutation such as a difference in species or strain of an organism, and the like. Also, they can be obtained by manuals of experiments on genetic recombination (Nucleic Acid Res. 10, pp. 6487 (1982) , Methods in Enzymol. 00, pp. 448 (1983)), PCR method (Molecular Cloning 2nd Edt., Cold Spring Harbor Laboratory Press (1989); PCR A Practical Approach IRL Press pp.200 (1991)).

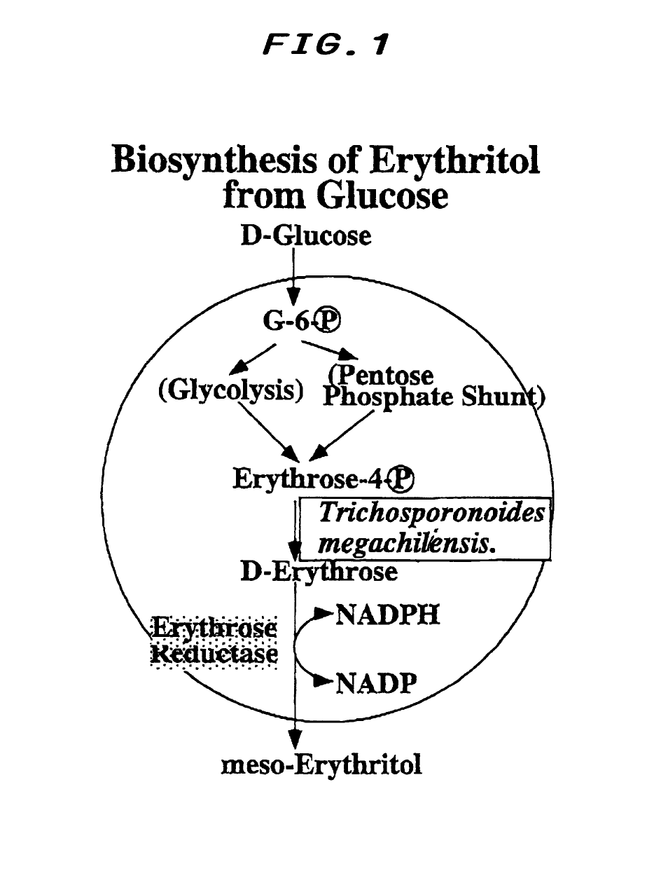

It can be confirmed by expressing the gene containing the sequence in a suitable cell and examining the erythrose reductase activity of the expression product whether said amino acid sequences containing the above-described substitution or the like have an erythrose reductase activity. The erythrose reductase activity can be measured by comparing changes in the amount of NAD(P)H since the enzyme uses NAD(P)H as a coenzyme upon reducing erythrose (cf. FIG. 1).

The DNA encoding the protein having an erythrose reductase activity of the present invention may be not only DNA containing the base sequence of base Nos. 1 to 398, which is, out of the base sequence described in SEQ. ID No. 1 in the Sequence Listing, on the N-terminal domain where it is predicted that the NAD (P) H binding site is mainly located but also DNA that hybridizes with a probe prepared from the above base sequence under stringent conditions and encodes a protein having an erythrose reductase activity.

Also, the DNA may be not only DNA containing the base sequence of base Nos. 408 to 1119, which is, out of the base sequence described in SEQ. ID No. 1 in the Sequence Listing, a portion on the C-terminal where the erythrose or erythritol binding site is present but also DNA that hybridizes with a probe prepared from the above base sequence under stringent conditions and encodes a protein having an erythrose reductase activity.

The “stringent condition” referred to herein means the condition where a so-called specific hybrid forms without non-specific hybrid.

While this condition is difficult to be described in numerical values, mention may be made, for example, the condition under which hybridization is performed at 60° C., 2×SSC, and 0.1% SDS, i.e., the condition of washing in usual Southern hybridization.

Among the DNA that hybridize under these conditions, those in which stop codon has been generated in the midway and those that have lost activity due to the variation in the active center may be included. They can be easily removed by ligating them to a commercially available expression vector, expressing them in a suitable host and measuring the erythrose reductase activity of the expressed product by the method described hereinbelow.

The cDNA encoding the erythrose reductase can be introduced into a cell in the form in which the erythrose reductase expresses its activity.

The introduction into a cell can be performed by amplifying by PCR the full length of cDNA encoding the erythrose reductase with a primer having a restriction enzyme recognition site at both ends and ligation the amplified DNA into various expression vectors at the restriction enzyme site.

The cells, such as Escherichia coli, yeast (Saccharomyces cerevisiae and Pichia pastoris) and the like can be used for erythrose reductase expression.

As the plasmid, it is desirable to select suitable expression vectors for large scale production. In the case of Escherichia coli, plasmids encoding histidine tag-fused protein, GST (Glutathione-S-transferase)-fused protein, thioredoxin-fused protein and the like can be used.

Upon the induction of expression, a promoter may be incorporated upstream of the 5′ side and a terminator may be incorporated downstream of the 3′ side of the DNA of the present invention. As the promoter and terminator, those that are known to have a function in the cell for expression must be used. Details thereof are described in Biseibutsugaku Kisokoza 8, Idenshikogaku, Kyoritsu Shuppan (Fundamental Course on Microbiology 8, Genetic Engineering, Kyoritsu Print), Adv. Biochem. Eng. 43, 75-102 (1990), Yeast 8, 423-488 (1992) and the like.

For example, where a plasmid is introduced into Escherichia coli, use of a regulation system by IPTG (isopropyl thiogalactopyranosid) at lactose operon and Lac I is preferable for tightly regulated induction.

As described above, the protein having an erythrose reductase activity of the present invention can be produced by culturing cells to which DNA encoding a protein having an erythrose reductase activity is introduced, performing IPTG induction, harvesting the cells at a stage when the protein sufficiently expresses, separating recombinant proteins and purifying them.

Taking an example, a recombinant erythrose reductase can be produced by harvesting the protein expressing cells by centrifugation, crushing the cells by ultra-sonication, purifying the obtained recombinant proteins by affinity gel chromatography with histidine tag, and cleaving the histidine-tag off with enterokinase.

The erythrose reductase activity can be monitored by measuring absorbance change at 340 nm in accordance with NADPH consumption upon reducing D-erythrose (cf. FIG. 1).

Next, the method for producing erythritol with the present invention will be described. According to the first method, meso-erythritol can be obtained by making the protein of the present invention act on D-erythrose in the presence of NA(D)PH.

Upon making the protein having erythrose reductase activity of the present invention reduce D-erythrose in the presence of NA(D)PH, at an optional condition for the enzyme activity.

The reduction reaction can be performed at a reaction temperature of 33 to 37° C., preferably 36 to 37° C., pH 6.0 to 7.0, preferably pH 6.5, in a coenzyme NADPH concentration of 0.1 to 0.5 mM, preferably 0.2 to 0.3 mM. The substrates can be added at the starting point for the reaction but it is desirable to continuously or non-continuously add the substrate so that the concentration of substrate in the reaction mixture will not become too high.

Also, erythritol can be obtained by making the cell to which the DNA encoding an erythrose reductase protein has been introduced on D-erythrose. In this case, erythrose reductase protein reduces D-erythrose, using intercellular NADPH.

Taking an example, erythritol can be obtained by culturing the cells to which the DNA encoding erythrose reductase protein is incorporated under a suitable condition for the growth of the cells and the production of erythritol in a medium containing properly a carbon source selected from sugars such as glucose, fructose, sucrose and the like, a nitrogen source selected from yeast extracts, peptone and the like, and an inorganic salts selected from phosphoric acid salts, magnesium salts, calcium salts, etc.

The recombinant erythrose reductases of the present invention has a substrate specificity equivalent to that of the erythrose reductases reported and has an enzymatic activity of producing sugar alcohol.

According to the present invention, a novel protein having an erythrose reductase activity can be provided.

Expression of DNA encoding the erythrose reductases of the present invention, for example, yeast cells and the like make it easy to perform large scale production of erythrose reductases independently of the productivity of the yeasts and the like as compared with the known methods by culturing yeast.

The recombinant erythrose reductases have equivalent substrate specificity to that of natural type erythrose reductases and also retain an enzymatic activity of producing sugar alcohols.

Therefore, the erythrose reductases produced by utilizing DNA encoding the enzyme of the present invention are useful in the production of erythritol on an industrial scale.

Moreover, DNA encoding the erythrose reductases of the present invention is also useful in various applications such as development of mutant enzymes having high erythritol productivity by genetic engineering techniques and cloning of genes encoding related enzymes.

### EXAMPLES

Hereinafter, the present invention will be explained in detail with examples. However, the present invention should not be construed as being limited thereto.

### Example 1

(1) Harvesting and Purification of Erythrose Reductase type I, II, III from Trichosporonoides megachiliensis Strain SN-G42

Following the method described in H. Ishizuka, et al., Biosci. Biotech. Biochem., 56(6), 941-945, 1992, harvesting and purification of erythrose reductases type I, II, III from Trichosporonoides megachiliensis strain SN-G42 (FERM BP-1430, under the old name of Aureobasidium strain SN-G42; this strain is deposited under the Budapest Treaty on the National Institute of Bioscience and Human Technology (NIBH), Agency of Industrial Science and Technology, Ministry of International Trade and Industry) was performed.

First, Trichosporonoides megachiliensis Strain SN-G42 (FERM BP-1430) was cultured for 72 hours in glucose medium (40% glucose, 2% yeast extracts, 2 liters).

The cells were collected by centrifugation at 10,000×g for 30 minutes then freeze-dried, treated with acetone, and thereafter homogenized using MSK Cell homogenizer (produced by B. Braun Japan).

Next, the crushed cells were centrifuged at 10,000×g for 30 minutes in 4° C. to remove cell debris.

The supernatant was subsequently fractionated by ammonium sulfate precipitation. The precipitants between 40 to 70% ammonium sulfate including erythrose reductase were condensed by membrane filtration. After removing insoluble materials by centrifugation, the enzyme fraction was dialyzed against the above-described buffer solution at 4° C. for 24 hours.

The dialyzed sample was loaded on a column of DEAE-Toyopearl 650S (1.4 20 cm) (produced by Tosoh Corp.) previously equilibrated with 50 mM glycine-NaOH buffer solution (pH 9.0) and the concentration of sodium chloride was linearly increased from 0 mM to 100 mM, followed by recovering the fractions containing erythrose reductase activity.

The active fractions were condensed by ammonium sulfate precipitation and loaded on a column of AF-Blue Toyopearl 650ML (1.4×20 cm) (produced by Tosoh Corp.) previously equilibrated with 10 mM phosphate buffer solution (pH 6.0) and the concentration of sodium chloride was increased stepwise from 0 mM to 200 mM, followed by separating the fractions containing erythrose reductases type I and type II (non-adsorbed fractions) and fractions containing erythrose reductase type III (adsorbed fractions).

After collecting and condensing them, the fractions containing erythrose reductase type III were loaded on a column of Butyl-Toyopearl 650S (11×20 cm) (produced by Tosoh Corp.), a column for hydrophobic chromatography previously equilibrated with 35% saturated ammonium sulfate-10 mM phosphate buffer solution (pH 6.0), and 10 mM phosphate buffer solution (pH 6.0) was passed under a gradient of concentration of ammonium sulfate linearly descending from 35% to 20% to recover the fractions having erythrose reductase activity.

Thus, erythrose reductase type III was purified.

(2) Determination of a partial amino acid sequence of erythrose reductase type III

Peptide mapping of the above-purified erythrose reductase type III was performed to determine a partial amino acid sequence.

The erythrose reductase type III was pyridylethylated (H. Hirano: J. Protein Chem., 8, 115 (1989)) and digested with lysyl endopeptidase (produced by Roche). Separation of this sample using ODS-80Tm (produced by Tosoh Corp.) column showed 14 peaks, two of which were determined for amino acid sequence using Peptide Sequencer 477A (produced by Perkin-Elmer).

(3) Design of a primer used in PCR reaction

Of the partially decoded amino acid sequence, those amino acid sequences (cf. SEQ. ID Nos.4 and 5 in the Sequence Listing) selected with reference to the amino acid sequences in among aldo-keto reductase family were used as a sense primer (cf. SEQ. ID No. 2 in the Sequence Listing) and an antisense primer (cf. SEQ. ID No. 3 in the Sequence Listing) in the PCR reaction described hereinbelow.

(4) PCR of cDNA fragment encoding erythrose reductase type III

Next, to prepare a probe for use in the screening and the like as described below, PCR was performed.

Single strand cDNA was synthesized from Trichosporonoides megachiliensis strain SN-G42 cultured for 3 days in 40% glucose medium by the following procedures and used as a template.

RNA was extracted from the culture cell using TRIZOL (produced by Gibco BRL) and mRNA was purified using a DYNABEADS mRNA Purification Kit (produced by DYNAL).

Reverse transcription reaction was carried out using the purified mRNA as a template to synthesize cDNA.

Super Script™ Reverse Transcriptase (produced by Gibco) was used as a reverse transcriptase and Oligo(dT) ₁₂₋₁₅ primer (produced by Amersham Pharmacia Biotech) as a primer.

The composition of the reverse transcription reaction mixture was as follows:

| | | |
|---|---|---|
| mRNA | 1 | μg |
| dNTP | 10 mM × 3 | μL |
| Primer | 0.5 | μg |

RNA transcription was carried out at 42° C. for 1 hour.

Using the thus-obtained cDNA as a template, the sense primer (cf. SEQ. ID No. 2 in the Sequence Listing) and antisense primer (cf. SEQ. ID No. 3 in the Sequence Listing) for PCR designed in (3) above, and Pfu DNA polymerase (produced by STRATAGENE), PCR reaction was carried out 25 cycles, each cycle being 94° C., 1 minute −40° C., 1 minute −72° C., 1 minute.

The amplification product of PCR reaction was a cDNA fragment of a length of 398 bp. The fragment was ligated to a vector pBS SK+ digested with Eco RV and further was transformed with DH5α strain. Whether or not the transformant contained the inner amino acid sequence of the previously determined erythrose reductase type III protein was analyzed.

The cDNA fragment obtained was consequently identified as being a partial cDNA of erythrose reductase type III. This fragment corresponded to 184 th to 582nd from the N-terminal of the base sequence described in SEQ. ID No. 1 in the Sequence Listing.

This cDNA fragment was used as a probe for the further cDNA isolation.

(5) Northern hybridization of Trichosporonoides megachiliensis strain SN-G42

Upon preparing cDNA library of Trichosporonoides megachiliensis strain SN-G42, Northern hybridization was performed in order to study when the microorganism express mRNA for erythrose reductase type III.

Trichosporonoides megachiliensis Strain SN-G42 was cultured with shaking under the condition of 37° C. and 220 rpm in a 500 ml flask using 30 ml of a medium containing 40% glucose for 24, 48, 72 or 96 hours.

After the culture, total RNA was extracted from each cells using TRIZOL (produced by Gibco BRL).

The probe prepared from the previously decoded purified enzyme was used after labeling with digoxigenin-UTP using DIG RNA Labeling kit (produced by Roche).

Extracted RNAs was electrophoresed, then blotted onto a Hybond-N membrane. Northern hybridization was carried out with the above RNA-labeled probe under high stringent condition.

FIG. 2 shows the results of Northern hybridization. In FIG. 2, Lane 1 shows the results on the product of 24 hours culture, Lane 2; 48 hours, Lane 3; 72 hours and Lane 4; 96 hours. In order to compare the lengths of fragments, the mobility of RNA Ladder was marked on the left side.

From FIG. 2, the band of around 1.0 kb was strongest at 48 hour culture.

From these results, it is clear that the 48 hour culture of Trichosporonoides megachiliensis strain SN-G42 expressed erythrose reductase type III highest. This reveals that the cDNA library prepared from RNA at this time is suitable for gene analysis of the above-described enzyme.

(6) Preparation of cDNA library of Trichosporonoides megachiliensis strain SN-G42

In accordance with the results in (5) above, a cDNA library was prepared from mRNA at the time when erythrose reductase type III expressed highest by the following procedure.

Trichosporonoides megachiliensis strain SN-G42 was cultivated in 30 ml of a medium containing 40% glucose in a 500 ml flask under the conditions of 37° C., 220 rpm and 48 hours.

RNA was extracted from the culture using TRIZOL (produced by Gibco BRL) and mRNA was purified using DYNABEADS mRNA Purification Kit (produced by DYNAL).

From the mRNA, a library was prepared using ZAP Express cDNA Synthesis Kit (produced by STRATAGENE) with the description of the kit. First, reverse transcription reaction was carried out using mRNA as a template to synthesize cDNA.

On this occasion, Moloney murine leukemia virus reverse transcriptase (MMLV-RT, produced by STRATAGENE) was used as a reverse transcriptase and linker primer was used as a primer. These are reagents contained in the above-described kit.

The composition of reverse transcription reaction mixture was as follows.

| | | |
|---|---|---|
| mRNA | 5 | μg |
| dNTP | 10 mM × 3 | μL |
| Primer | 2.8 | μg |

The obtained cDNA was inserted into ZAP Express Vector utilizing EcoR I site and Xho I site to package it.

In this manner, the cDNA library of Trichosporonoides megachiliensis strain SN-G42 having a titer of 2,350,000 pfu was prepared.

(7) Plaque hybridization of cDNA library and Determination of Base Sequence of DNA encoding an Erythrose Reductase Type III Protein

Next, packaged recombinant phage was infected to Escherichia coli XLI-Blue MRF′ and was allowed to form plaques on the plate. Then, using the probe prepared in (4) above, plaque hybridization was carried out under the stringent conditions.

As a result of the plaque hybridization, the target clone was isolated and amplified and thereafter by acting helper-phage, only the phagemid portion in the λ-phage DNA (including an insert) was synthesized and cyclized to form a plasmid. This was infected to host Escherichia coli XLOLR and amplified therein.

Then, a plasmid was obtained from the amplified XLOLR and DNA sequencing was performed.

As a result of analyses, this revealed to be a base sequence described in SEQ. ID No. 1 in the Sequence Listing of a full length of 1,119 bp. The translation of the base sequence into amino acid was also shown in SEQ. ID No. 1 in the Sequence Listing.

Furthermore, a restriction enzyme map on the obtained base sequence was prepared by the conventional manner (FIG. 3). As will be apparent from FIG. 3, the base sequence has a Ban I cleavage site on the 122nd base portion counted from the 5′-terminal, EcoR I cleavage sites in the 847th and 1057th base portions and BamH I cleavage site on the 1093rd base portion.

The erythrose reductase type III protein consisting of the amino acid sequence described in SEQ. ID No. 1 in the Sequence Listing revealed to be a novel sequence having low homology with the known amino acid sequences such as the previously elucidated human aldose reductase enzyme and yeast gcy protein.

From this it revealed that the DNA encoding the erythrose reductase type III protein of the present invention has the sequence described in SEQ. ID No. 1 so that the erythrose reductase type III gene of the present invention revealed to be a polypeptide having a novel amino acid sequence.

(8) Expression of DNA Encoding Erythrose Reductase Type III Protein

Based on the N-terminal side of the base sequence described in SEQ. ID No. 1 in the Sequence Listing 1, a primer was prepared so as to have a BamH I site. Also, based on the C-terminal side of the same base sequence, another primer was synthesized so as to have an Xho I site.

Screening from the cDNA library was performed to obtain a plasmid containing erythrose reductase type III. Using this as a template, full-length cDNA of erythrose reductase type III having BamH I and Xho I sites on the terminals was amplified by PCR using the above-described primers.

The conditions of PCR were such that 200 ng of plasmid containing erythrose reductase type III cDNA was used as a template and Pfu polymerase (produced by STRATAGENE) was used as an enzyme and PCR was carried out at 12 cycles.

As a result of PCR, erythrose reductase type III gene having BamH I, Xho I sites was amplified. The PCR amplification product was incorporated by cleaving the BamH I and Xho I sites of plasmid pRSETA (produced by INVITROGEN).

The plasmid PRSETA in a state having incorporated therein erythrose reductase type III gene was introduced into Escherichia coli BL21 (DE3) pLysS (prepared by STRATAGENE) and Escherichia coli was cultivated at 25° C. so that it can be expressed as histidine Tag fused protein. The induction of expression was performed by adding IPTG at a final concentration of 1 mM through lactose operon.

Esherichia coli cells were collected by centrifugation (2,920×g, 15 minutes) and crushed by sonication treatment (SONIFIER 250D, produced by BRANSON) and again centrifugation (26,400×g, 15 minutes) was performed to obtain supernatant (crude enzyme solution).

The obtained crude enzyme solution was purified through Nickel Chelated Agarose (B-PER 6×His Spin Purification Kit, produced by PIERCE), which is an affinity gel for a histidine Tag-fused protein.

Next, using a gel filtration unit PD-10 (produced by Amersham Pharmacia Biotech), the buffer was replaced with an enterokinase reaction buffer using gel filtration.

After the purification, the Tag (peptide) portion was cleaved with enterokinase (produced by INVITROGEN). Thereafter, to remove contaminant proteins, Tag, and enterokinase, purification by ion exchange column was performed to obtain recombinant erythrose reductase type III.

(9) Activity of Recombinant Erythrose Reductase Type III

The enzymatic activity of recombinant erythrose reductase type III was measured.

The enzyme was acted in the presence of 12 mM of a substrate, 0.2 mM of NADPH and a change in optical absorbance at 340 nm was measured with time for 10 minutes.

The enzymatic activity was defined 1 unit when 1 μmol of NADPH is oxidized for 1 minute. The amount of oxidized NADPH was calculated from the decreased initial speed in a value measured at 340 nm and the unit number calculated from the amount of liquid used for the measurement into the amount of liquid of total sample was defined as total activity. Further, total activity was divided by the amount of protein (mg) contained in the sample, and thus obtained value was defined as specific activity (units/mg).

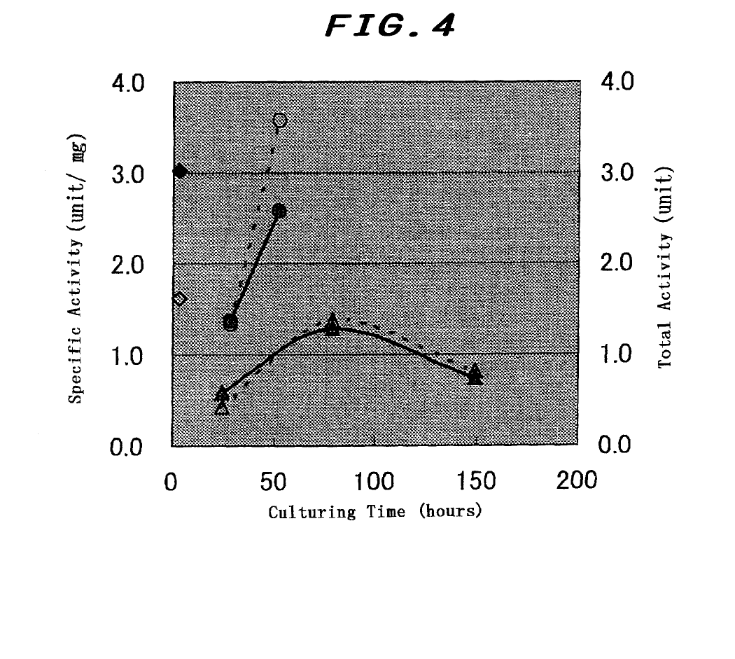

To confirm the optimum temperature for induction of expression, induction of expression was performed at 37° C., 25° C., or 20° C. after the addition of IPTG, and samples purified by nickel chelated agarose were measured of activity. The results are shown in FIG. 4.

In FIG. 4, ♦, ●, and ▴ stand for specific activities (units/mg) at 370° C., 25° C., and 20° C., respectively and ⋄, ◯, and Δ stand for total activities (unit) at 37° C., 25° C., and 20° C., respectively.

From FIG. 4, it can be seen that the purified recombinant erythrose reductase type III of the present invention is expressed in any of 20° C., 25° C. and 37° C., and that in particular it is expressed in high efficiency at 25° C.

Furthermore, comparison was made in substrate specificity between the purified recombinant erythrose reductase type III and the erythrose reductase heretofore reported (natural type: H. Ishizuka, et al., Biosci. Biotech. Biochem., 56(6), 941-945, 1992).

The reaction rate where erythrose is reduced as a substrate is taken 100% and relative values (%) of reaction rates for reducing various ketoses and aldoses are shown in Table 1.

TABLE 1

Relative Value (%)

| substrate | Natural Type | Recombinant Type |
|---|---|---|
| Dihydroxyacetone | 20.0 | 9.0 |
| D-glyceraldehyde | 66.0 | 81.2 |
| D-erythrose | 100.0 | 100.0 |
| L-erythrulose | 38.0 | N.D |
| D-ribose | 1.2 | 2.7 |
| D-arabinose | 0.0 | 0.8 |
| D-xylose | 1.2 | 5.1 |
| D-xylulose | 0.0 | N.D |
| D-glucose | 0.0 | 0.6 |
| D-mannose | 0.0 | N.D |
| D-galactose | 0.0 | 0.3 |
| D-fructose | 0.0 | N.D |
| L-sorbose | 0.0 | N.D |
| Trehalose | 0.0 | N.D |
| D-glucuronate | 6.6 | 1.5 |
| p-nitrobenzaldehyde | 46.0 | 84.5 |

From Table 1, it is clear that the purified recombinant erythrose reductase of the present invention has an ability of reducing substrates of heretofore reported natural type erythrose reductase type III.

From the above, it is apparent that the recombinant enzyme obtained by the expression of DNA encoding the erythrose reductase type III of the present invention has the same substrate specificity as the naturally occurring erythrose reductase type III and has an enzymatic activity of producing sugar alcohol.

What is claimed is:

1. A protein shown in (A) or (B) below:
(A) a protein having an amino acid sequence of SEQ. ID No. 1 in the Sequence Listing;
(B) a protein having an amino acid sequence of SEQ. ID No. 1 in the Sequence Listing, wherein the amino acid sequence includes substitution, deletion, insertion, addition or inversion of one or several amino acids and wherein the protein has an erythrose reductase activity.

2. A DNA encoding a protein shown in (A) or (B) below: (A) a protein having an amino acid sequence of SEQ. ID No. 1 in the Sequence Listing;
(B) a protein having an amino acid sequence of SEQ. ID No. 1 in the Sequence Listing, wherein the amino acid sequence includes substitution, deletion, insertion, addition or inversion of one or several amino acids and wherein the protein has an erythrose reductase activity.

3. The DNA as claimed in claim 2, wherein the DNA comprises one shown in (a) or (b) below:
(a) a DNA containing a base sequence comprising at least nucleotides Nos. 1 to 398 out of the nucleotide sequence described in SEQ. ID No. 1 in the Sequence Listing.
(b) a DNA hybridizing with a base sequence comprising at least nucleotides Nos. 1 to 398 out of the nucleotide sequence described in SEQ. ID No. 1 in the Sequence Listing or a probe prepared therefrom under a stringent condition and encoding a protein having an erythrose reductase activity.

4. The DNA as claimed in claim 3, wherein the stringent condition is a condition under which washing is performed at a salt concentration corresponding to 2×SSC and 0.1% SDS at 60° C.

5. The DNA as claimed in claim 2, wherein the DNA comprises a DNA shown in (c) or (d) below:
(c) a DNA containing a base sequence comprising at least nucleotides Nos. 408 to 1119 out of the nucleotide sequence described in SEQ. ID No. 1 in the Sequence Listing.
(d) a DNA hybridizing with a base sequence comprising at least nucleotides Nos. 408 to 1119 out of the nucleotide sequence described in SEQ. ID No.1 in the Sequence Listing or a probe prepared therefrom under a stringent condition and encoding a protein having an erythrose reductase activity.

6. The DNA as claimed in claim 5, wherein the stringent condition is a condition under which washing is performed at a salt concentration corresponding to 2×SSC and 0.1% SDS at 60° C.

7. A cell to which a DNA has been transferred as claimed in any one of claims 2 to 6 in a manner such that the DNA is capable of expressing an erythrose reductase type III the DNA encodes.

8. A method for producing erythrose reductase type III, comprising the steps of cultivating a cell as claimed in claim 7 in a medium to produce and accumulate erythrose reductase type III in a culture liquid and harvesting the erythrose reductase type III from the culture liquid.

9. A method for producing erythritol, comprising the steps of acting the protein having an erythrose reductase activity as claimed in claim 1 on D-erythrose and harvesting a produced erythritol.

10. A method for producing erythritol, comprising the steps of acting the cell as claimed in claim 7 on D-erythrose and harvesting a produced erythritol.
